(19) United States
(12) Patent Application Publication
(10) Pub. No.: US 2020/0253960 A1
(43) Pub. Date: Aug. 13, 2020

(54) ANTIBIOTIC FORMULATIONS FOR TREATING OF CAVITY INFECTIONS

(71) Applicant: ARADIGM CORPORATION, Hayward, CA (US)

(72) Inventors: Igor GONDA, San Francisco, CA (US); John Siebert, Kailua Kona, HI (US)

(73) Assignee: ARADIGM CORPORATION, Hayward, CA (US)

(21) Appl. No.: 16/748,472

(22) Filed: Jan. 21, 2020

Related U.S. Application Data
(60) Provisional application No. 62/803,983, filed on Feb. 11, 2019.
(60) Provisional application No. 62/930,884, filed on Nov. 5, 2019.

(51) Int. Cl. A61K 31/496; A61K 9/127

(52) U.S. Cl. CPC A61K 31/496; G01N 33/56911; A61K 9/127

(57) ABSTRACT

A method of treating a subject, comprising: (a) diagnosing a subject with bronchiectasis and chronic respiratory bacterial infections, with at least one strain of a bacteria such as Pseudomonas aeruginosa bacteria in an airway sample taken from the subject; (b) administering to the subject over a plurality of days (such as 28 days) by inhalation a formulation comprised of the un-encapsulated and encapsulated antibiotic, such as ciprofloxacin; (c) discontinuing the administering over a plurality of days; (d) repeating the administering over a plurality of days; (e) whereby an antibacterial impact of the formulation on the bacteria is not decreased with each repeating step (d).

### FIELD OF THE INVENTION

The present invention relates generally to methods of treatment and particularly to methods of treating refractory infections in body cavities including lung infections, wherein the method involves repeatedly administering a particular formulation without a decreased impact on bacteria treated, particularly on treating a patient population shown to be resistant to treatment for an infection due to specific criteria outlined here.

### BACKGROUND TO THE INVENTION

Antibiotic drugs are developed to hit targets with the view to kill or inhibit the growth of microorganisms that might be causing pathological effects in humans or animals. Antibiotic drugs that are effective against bacteria are also called “antibacterials”. Antibiotics belong to the general class of anti-infectives encompassing antibacterials, antibiotics, antifungals, antiprotozoans and antivirals. Unfortunately, these drugs are rarely without side-effects—in other words, they do impact mammalian cells including humans as well which may cause a variety of side-effects. The therapy with antibiotics is therefore limited to doses that are deemed to be safe and tolerable to mammals based on experimental data during the development of the antibiotic and the post-approval surveillance.

Resistance to an antibiotic is the ability of a bacterium to withstand the effects of this drug. It is a specific type of drug resistance. Antibiotic resistance may evolve from several causes including random mutation, but it could also be activated by applying an evolutionary stress on a population or through transmissions of plasmids and so on.

Antibiotic resistance occurs when an antibiotic has lost its ability to effectively kill bacteria or stop bacterial growth at concentrations that are clinically achievable at the site of the infection in a human being or animal; in other words, the bacteria are “resistant” and continue to multiply in the presence of the drug at the concentrations that are typically limited by the safety and tolerability of the drug in those parts of the body that the antibiotic reaches. Typically, the degree of drug resistance (or its opposite, susceptibility) is given as the minimum concentration (MIC) of the antibiotic that inhibits the growth of that particular organism; if that concentration is higher than the maximum safe and tolerable concentration (i.e., clinically achievable concentration), then the bacterium is deemed to be “resistant” to that particular drug because higher doses resulting in higher concentrations would cause side-effects. Examples of such side-effects are nephrotoxicity and ototoxicity caused by aminoglycoside antibiotics; central-nervous system side effects caused by, e.g., fluoroquinolones. Therefore, increasing the doses of antibiotic drugs using the same route of administration is not a viable strategy to overcome resistance.

Antibiotic drugs are typically approved with specified maximum daily doses which may be further limited for specific populations, such as for the very young or the very old, or for people with pre-existing conditions that put them at a higher risk of side-effects.

Orally administered antibiotics often disturb the normal bacterial flora of the gastrointestinal tract which may then result in a variety of side-effects including diarrhea. Colonization of the gastrointestinal tract with pathogenic opportunistic infections such as Clostridium difficile could be a serious complication resulting from long-term use of oral antibiotics.

The resistance to antibiotic therapies has become a global threat. It is important to understand the series of events that have led the world to this predicament. Originally touted as a miracle discovery, we are seeing that there can be “too much of a good thing” with antibiotics.

So, while antibiotics have saved millions of lives, their pervasive use to treat any infection, whether serious, minor, or even incorrectly diagnosed such as treating a viral infection with an antibacterial drug, has led to the increase in antibiotic resistance.

Antibiotics developed to target specific microorganisms may not be effective against other organisms or, indeed, could make these organisms drug-resistant if used at sub-optimal doses. However, identifying the nature of the infection and its susceptibility to a particular microorganism is not always done because of time-delays that could have serious impact on the patient's well-being and costs associated with the testing. Anti-bacterials should not be used against viral infections but, again, delaying the decision to treat before the cause of the infection is known may be risky and the tests to determine it could be time-consuming and costly.

Another issue with antibiotic use is the patient's compliance. Antibiotic dosage regimens including the duration of the treatment are designed to eradicate the pathogens or significantly reduce their load to a level such that the patient's body (immune system) can eliminate any remaining infection. When the patient is not compliant with the prescribed dosage regimen, for example does not take the drug at the prescribed time and for a prescribed period, pathogenic bacteria may adapt to the presence of these drugs, and eventually form a population that is resistant to the drug even at higher doses and could be treated only at doses causing significant side-effects.

Antibiotic usage is not exclusive to humans. Every day, several types of these drugs are used to treat livestock and fish to prevent infections. Similar to overuse in humans, inappropriate use of antibiotics creates a reservoir of bacteria that could be resistant, thus rendering these drugs useless in animals and potentially also in humans if these bacteria are transmitted to humans.

As a result of cities becoming more densely populated, people are exposed to more pathogens all the time. Hospitals and clinics are seeing more and more patients with infections, and it is not always possible to curb the spread of a pathogen in a population.

Identification, isolation or treatment of all infectious diseases are not often feasible, resulting in the addition of more pathogens to the local community. Coupled with lack or inadequate hygiene and poor sanitation, urban centers become and ideal breeding ground for bacteria.

The global resistance problem is also confounded by the slow arrival of new antibiotic therapies that could overcome the resistance development to already existing drugs. Following an unprecedented number of antibiotics in the 20th century, the number of newly approved antibiotics has slumped to an all-time low.

In summary, the 6 main causes of the global antibiotic resistance crisis have been linked to:
Over-prescription of antibiotics
Patients non-compliant with their dosage regimens
Overuse of antibiotics in livestock and fish farming
Poor infection control in health care settings
Poor hygiene and sanitation
Few new antibiotics entering the market

### SUMMARY OF THE INVENTION

An aspect of the invention is a particular formulation comprising both unencapsulated and encapsulated antibiotic which is delivered to a body cavity over a period of time using a particular dosage schedule which composition and schedule preserves the anti-bacterial effectiveness relative to particular infections over a prolonged period of time even when repeatedly administered at different points in time.

In an aspect of the invention, the antibacterial compound is ciprofloxacin which is present as a mixture of ciprofloxacin in solution and as ciprofloxacin encapsulated in liposomes. The mixture is administered on a daily basis for 28 days followed by no administration for 28 days, and again administering on a daily basis for 28 days without loss of anti-bacterial affect.

An aspect of the invention is that the formulation and dosing schedule used on a particular bacterial strain reaches the highest minimum inhibitory concentration (MIC) after the first cycle of dosing for 28 days which remains substantially unchanged when the formulation and dosing schedule are repeated such that any decrease in antibacterial impact is less than 10%, less than 5%, or less than 1%.

An aspect of the invention is a method of treatment wherein the patient treated is selected based on criteria which include a refractory infection previously treated unsuccessfully, wherein the patient is further determined to have a characteristic selected from the group consisting of (1) lack of compliance with prescribed dosing regimen; (2) improper inhalation technique; (3) problems with inhalation device used in treatment; (4) lack of drug stability and/or formulation stability; and (5) airway obstruction causing failure of formulation to enter lungs to cite of infection.

The invention is an inhalable antibiotic formulation that prevents or overcomes several problems associated with the use of orally or intravenously administered antibiotics. While the invention is focused on inhalation delivery, it is generally applicable to the delivery of the said formulations to the respiratory tract by methods other than inhalation, e.g., instillation, or to other body cavity infections.

The general design features of these formulations are that they comprise of one or more antibiotics which may be present entirely encapsulated, or as mixtures of unencapsulated (free) and encapsulated drugs. These formulations are then administered directly into the respiratory tract or another body cavity that has the infection, such as the ear.

Specific formulations and methods of administration are disclosed here. However, variations in the formulation as well as the dosing schedule can be expected provided the changes result in obtaining the substantially the same result in terms of a formulation and dosing schedule which when delivered maintain its anti-bacterial impact over time. The formulation will include an anti-bacterial agent, which may be ciprofloxacin. The ciprofloxacin will be present in solution at a concentration of about 10 to 40 mg/ml or about 20 to 30 mg/ml ±20%, ±10%, ±5%. The antibiotic such as the ciprofloxacin is also present inside of liposomes. In addition to the antibiotic solution and liposomes containing the antibiotic, the liposomes are formulated in a manner such that 90%, 95%, 98% or more of the liposomes maintain their structural integrity when aerosolized for delivery. The liposomes may be comprised of cholesterol and hydrogenated soy phosphatidyl choline (HSPC). In addition to the main active ingredients, secondary active ingredients may be present. Further, excipients may be present and include loading agents which aid in the encapsulation of the antibiotic inside of liposomes, such loading agents may include ammonium sulfate and methyl ammonium sulfate as well as other compounds known to those skilled in the art. The formulation may include pH adjusting agents such as sulfuric acid and sodium hydroxide in amounts needed to obtain the desired pH. Suitable buffers may be used to maintain the pH such as sodium acetate and histidine. Water is added along with salts such as sodium chloride in order to adjust the osmolality and tonicity of the formulation. Additional excipients may include glacial acidic acid and other substances to adjust the pH, osmolality, or taste, of the formulation.

The formulation may be delivered by various types of aerosols by inhalation to the respiratory tract, or directly to the lungs by other delivery methods such as instillation or insufflation of liquids, semisolid or solids; such methods may be used also to directly deliver the formulation to other body cavities including the ear or nose. The formulation may be delivered daily, twice daily or three times per day, every day for 5 days, 1 week, 2 weeks, 3 weeks, or 4 weeks. After administration, the drug use may be continued for 5 days, 1 week, 2 weeks 3 weeks, or 4 weeks followed by re-administration of the drug in accordance with the same dosing schedule per day over the same number of days. The data shown here demonstrate that the impact of the antibiotic on the bacteria remains substantially the same with each subsequent administration (dosing cycle) of the drug over time when the dosing schedule is once daily, 28 days on treatment, 28 days off treatment. Under some circumstances, the preferred regimen may be continuous dosing of the drug, without cycling of period of on treatment/off treatment. However, the key aspect of the invention remains as the sustained antibacterial action during each repeated dosing cycle.

“BE patient” is a patient that has bronchiectasis. Bronchiectasis is defined as an abnormal thickness of bronchial walls. It is now diagnosed primarily using high resolution (HR) computer assisted tomography (CAT) scanning (HRCAT), although a lot of cases would be picked up earlier with X-rays and CAT scans. The most common causes of bronchiectasis are TB (that is how it was originally discovered), non-TB mycobacteria (NTM) lung infections, chronic obstructive pulmonary diseases (COPD), cystic fibrosis (CF) and non-cystic fibrosis bronchiectasis (NCFBE, or sometime for short just “BE”).

The presence of NCFBE is essentially diagnosed by exclusion of the other causes and is often “idiopathic”, i.e., of unknown origin. The patients in the clinical trials described here were also diagnosed by CAT scans and exclusion of CF. The ORBIT trials described here admitted “COPD” patients as long as it could be reasonably assumed that it was not caused by smoking (they had to have less than 10 pack-years of smoking). Similarly, as long as they were not treated for active TB or active NTM lung infections, they were included in trials described here.

“Higher MIC” means higher minimum inhibitory concentration. MIC is the lowest concentration at which inhibition of the growth of the microorganism is observed. The MIC can be the highest safe concentration that patients can tolerate. Thus, any bacteria that can survive higher (higher than the highest safe concentration) concentrations of that antibiotic are deemed to be resistant against that antibiotic. “Higher” MIC can mean many different things depending on the context, but generally it is used to mean a MIC that is higher than the MIC for the “wild” strains of the organisms that were not previously exposed to that particular antibiotic. Once a patient begins treatment with an antibiotic, it is often observed that either through development of tolerance or by selection, the patient begins to carry less susceptible microorganisms, i.e., those microorganisms with higher MICs.

In the case of patients in clinical trials described here samples of bronchial liquids were obtained mainly by collecting spontaneously expectorated sputum, but in some patient' s deep throat swabs or bronchoalveolar lavage were used. Induced sputum collection could be also used. The growth of various microorganisms were then examined as a function of concentration of various antibiotics to determine the MICs. If those MICs were higher than those generally deemed to be MICs of bacteria susceptible to that particular antibiotic (these values are published regularly), then the bacteria were classified as “resistant” or not sensitive.

When a patient takes Linhaliq (a specific mixture of unencapsulated ciprofloxacin in solution plus liposome-encapsulated ciprofloxacin) correctly, a large reduction in the number of the important pathogens in their sputum is achieved, in particular Pseudomonas aeruginosa that are of the highest interest because their presence and their amount/concentration correlates with the severity of the disease and is a strong predictor of various signs of morbidity and mortality.

Generally speaking, any Pseudomonas aeruginosa (PA) with a MIC>4 microgram/mL would be deemed to be resistant to ciprofloxacin because that is about as high a level of ciprofloxacin in the blood stream as is safe. However, because concentrations of ciprofloxacin in the lung that are several orders of magnitude higher than 4 microgram/mL by inhalation were obtained with Linhaliq, the term “resistant” as defined by the inability of oral or iv ciprofloxacin to treat PA with MICs higher than 4 mcg/mL ceases to be meaningful with the treatment described here. Furthermore, results provided here show very high concentrations of ciprofloxacin sustained in the sputum over the whole 28 day period when patients are on treatment, and again in the next on treatment period and so on. So, even patients who started trials described here with such “resistant” PA responded well clinically to inhaled ciprofloxacin in Linhaliq. The resistant PA grows slowly and therefore is less virulent, i.e., may not be causing a lot of damage.

The results provided here show that there is also something else happening that is unexpected. Those patients who develop higher MICs of ciprofloxacin (i.e., the MIC of their PAs goes up during the treatment with Linhaliq compared to the MICs they had before they started the trial on our drug) actually appear to respond clinically better than those who are not developing higher MICs. Those patients who use Linhaliq correctly including using the correct breathing and dosing as per the directions, will get Linhaliq delivered into the lung, and they will wipe out the more susceptible PA first thereby leaving (in smaller quantities) PA with higher MICs. Furthermore, it is known that the concentration of PA in sputum correlates with the inflammatory load in the lung, i.e., the more PA, the more inflammation. Further, the more inflammation, the easier it is for the patients to get pulmonary exacerbations that are often triggered by viruses, or allergens because they have highly inflamed airways susceptible to such external attacks.

The clinical endpoint is the reduction of the risk of pulmonary exacerbations (first exacerbation and subsequent exacerbations). Linhaliq reduces the number of the virulent PA, which leads to the reduction of the inflammation, which in turn makes the patients less susceptible to develop pulmonary exacerbations. The other part of the explanation is that the strongest predictor of future exacerbations are past exacerbations, so Linhaliq intervenes in this vicious downward spiral of infection, inflammation, exacerbation leading to more infection, inflammation and exacerbations manifested by more frequent visits to doctors, hospitalization and premature death.

An aspect of the invention comprises a method of treatment, comprising diagnosing a subject with bronchiectasis and chronic respiratory bacterial infections, with at least one strain of Pseudomonas aeruginosa bacteria in an airway sample taken from the subject in the last 12 months characterized by a minimum inhibitory concentration (MIC) higher than the MIC of other Pseudomonas aeruginosa bacteria in the sample against a systemically administered antibiotic; administering to the subject by inhalation a formulation comprised of the un-encapsulated and encapsulated antibiotic.

In an aspect of the invention as described above, wherein the antibiotic is ciprofloxacin and the encapsulation is in liposomes.

An aspect of the invention as described above, further comprises repeating the administering each day for 28 days; discontinuing the administering for 28 days; and repeating the administering each day for an additional 28 days.

An aspect of the invention as described above, further comprises repeating administration whereby an antibacterial impact of the formulation on the Pseudomonas aeruginosa is not decreased with each administration.

An aspect of the invention as described above, wherein the subject has an immediate history of treatment with chronic macrolide therapy.

An aspect of the invention as described above, wherein the macrolide is selected from the group consisting of:

Azithromycin,

Clarithromycin,

Erythromycin,

Roxithromycin.

An aspect of the invention as described above, wherein the administering is carried out by aerosolizing the formulation and inhaling the aerosol in the patient's lungs, whereby 90% or more of the liposome maintain their structural integrity when aerosolized.

An aspect of the invention as described above, wherein the liposomes are comprised of cholesterol and hydrogenated soy phosphatidyl choline (HSPC).

An aspect of the invention as described above, where the repeating step is carried out until the respiratory infection with Pseudomonas aeruginosa is no longer detected in the patient for at least 2 months.

An aspect of the invention as described above, wherein the ciprofloxacin inside the liposomes is at a concentration of 50% or more higher than a concentration of the unencapsulated ciprofloxacin solution.

An aspect of the invention as described above, wherein the formulation further comprises a buffer selected from the group consisting of sodium acetate and histidine.

An aspect of the invention as described above, wherein the highest minimum inhibitory concentration (MIC) of ciprofloxacin to Pseudomonas aeruginosa remains substantially unchanged (less than 10% decrease, less than 5% decrease, less than 1% decrease) with each repeated administration thereby allowing dosing to remain unchanged in each administration.

An aspect of the invention as described above, wherein the ciprofloxacin is present in the liposomes at a concentration sufficiently high that ciprofloxacin precipitates out of solution, and the precipitate dissolves when delivered to a patient after the liposomes open.

An aspect of the invention is a method of treatment, comprising administering directly to the infection in the body cavity a formulation comprised of a solution of antibiotic having therein and an encapsulated antibiotic; repeating the administering each day for 28 days; discontinuing the administering for 28 days; and repeating the administering each day for an additional 28 days.

An aspect of the invention as described above, wherein the antibiotic is ciprofloxacin, and wherein the infection is caused by Pseudomonas aeruginosa.

An aspect of the invention as described above, wherein the encapsulation is in liposomes.

An aspect of the invention as described above, wherein the body cavity is the respiratory tract.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a graph showing changes in the number of colony forming units of Pseudomonas aeruginosa in the sputum obtained for both a placebo and Tobi (inhaled tobramycin) cystic fibrosis treatment groups over a period of 24 weeks (TOBI Package Insert, 2015).

FIGS. 2A and 2B are each graphs showing changes in the number of colony forming units of Pseudomonas aeruginosa in the sputum of NCFBE patients obtained with treatment with AZLI (inhaled aztreonam) or placebo (Barker et at., 2014).

FIG. 3 shows the change in sputum density of Pseudomonas aeruginosa (log10 cfu/g) from baseline through cycle 6 of Arikace (inhaled liposomal ciprofloxacin). Each set of three bars is the change in sputum P aeruginosa density compared with baseline (day 1 of cycle 1) for days 1 (white), 14 (gray), and 28 (black) of each respective Arikace cycle. *p=0.003 for change in cfu across all of the Arikace treatment cycles relative to baseline (cycle 1, day 1). (Clancy et al., 2013).

FIG. 4 is a graph showing the changes in the density (colony forming units, cfus) of Pseudomonas aeruginosa in the sputum of NCFBE patients treated with Linhaliq (inhaled mixture of unencapsulated and liposomal ciprofloxacin) or placebo over a period of 48 weeks in the two trials ORBIT-3 and ORBIT-4.

FIG. 5 is a graph showing relative risks of getting an exacerbation depending on either the baseline MIC of Pseudomonas aeruginosa, or the highest MIC emerging during the in the 48 week ORBIT-3 and ORBIT-4 trials.

FIG. 6 is a graph showing the risk ratio of developing pulmonary exacerbations (PE) in inhaled Linhaliq (mixture of unencapsulated and encapsulated ciprofloxacin) vs placebo treated NCFBE patients stratified according to the reduction in the colony forming units of Pseudomonas aeruginosa in their sputum in the 48 week ORBIT-3 and ORBIT-4 trials.

FIG. 7 provides results of pooled data for the hazard ratio of the first pulmonary exacerbation in inhaled Linhaliq (mixture of unencapsulated and encapsulated ciprofloxacin) vs placebo treated NCFBE patients stratified according to their use of oral macrolide therapy at baseline in the 48 week ORBIT-3 and ORBIT-4 trials.

FIG. 8 provides results of pooled data for the risk ratio of pulmonary exacerbations in inhaled Linhaliq (mixture of unencapsulated and encapsulated ciprofloxacin) vs placebo treated NCFBE patients stratified according to their use of oral macrolide therapy at baseline in the 48 week ORBIT-3 and ORBIT-4 trials.

FIG. 9 shows the use of concomitant drugs in the inhaled Linhaliq (mixture of unencapsulated and encapsulated ciprofloxacin) vs placebo treated NCFBE patients in the 48 week ORBIT-3 and ORBIT-4 trials.

FIG. 10 shows the changes in the antibiotic susceptibility (MIC) to various antibiotics for Pseudomonas aeruginosa in the 48 week ORBIT-3 and ORBIT-4 trials in which inhaled Linhaliq (mixture of unencapsulated plus liposome ciprofloxacin) was compared to placebo in NCFBE patients.

FIG. 11 showing the pooled data changes in the highest ciprofloxacin MIC of Pseudomonas aeruginosa in respiratory samples during each 28 day cycle on treatment and 28 day cycle off treatment of NCFBE patients in the ORBIT-3 and ORBIT-4 studies, both for the placebo and Linhaliq groups.

FIG. 12 shows sputum and plasma concentrations of ciprofloxacin (solid lines) following once daily dosing of inhaled ciprofloxacin, compared to the maximum plasma and sputum concentrations of ciprofloxacin from the recommended doses of oral ciprofloxacin (broken lines).

### DETAILED DESCRIPTION OF THE INVENTION

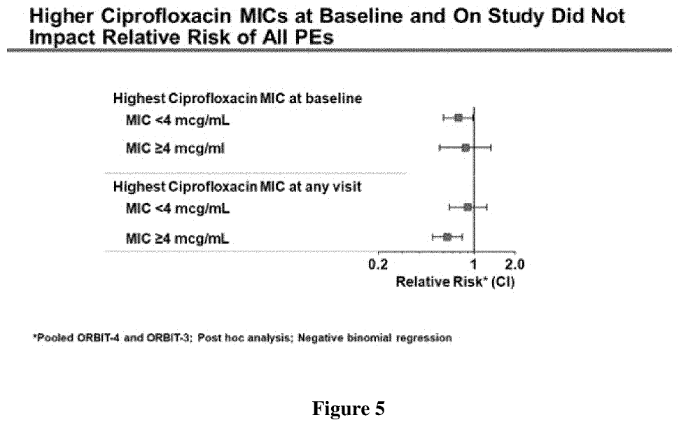

Surprisingly, some unexpected patient populations benefited more than others. For example, it is surprising and unexpected that BE patients with P. aeruginosa pulmonary infections who develop colonies with higher MICs (greater resistance to ciprofloxacin) on the inhaled therapy using the formulation and method of this invention actually did better than those who did not develop higher MICs. FIG. 5 shows that those who developed PA colonies with MICs above 4 mcg/mL (considered the point that resistance has developed for systemically administered ciprofloxacin) have better response to the Linhaliq treatment than patients who did not develop MICs above 4 mcg/mL.

It has been appreciated for some time that administering antibiotics by inhalation results in the desirable high concentration of the drug at the desired site of action—e.g., infections in the respiratory tract. Lower doses can be delivered that achieve much higher local concentrations than even much higher doses of oral or intravenous antibiotics. This then impacts the definition of “resistance”, or “susceptibility” which depends then on the route of administration. For example, the Spanish Antibiogram Committee has recommended specific resistance breakpoints for inhaled tobramycin against P. aeruginosa that differ from breakpoints for systemic therapy (breakpoint is the minimum inhibitory concentration for an antibiotic above which the microorganism is deemed to be resistant, i.e., e.g., the MIC). This group recommends that the resistant breakpoint be set at for inhaled tobramycin at 128 mg/liter, compared to the Clinical and Laboratory Standards Institute (CLSI) resistance breakpoint of 16 mg/liter for systemically administered tobramycin. (Inhaled Antibiotics for Gram-Negative Respiratory Infections Eric Wenzler, Dustin R. Fraidenburg, Tonya Scardina, Larry H. Danziger, Clin. Microbiol. Rev. 2016 Volume 29 Number 3 p.581).

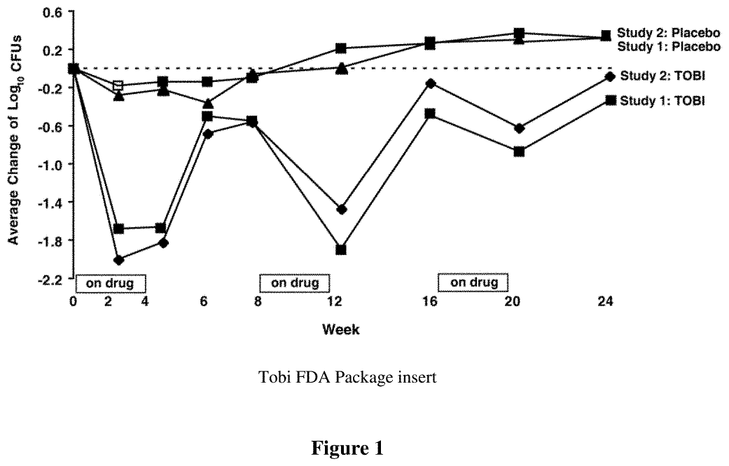

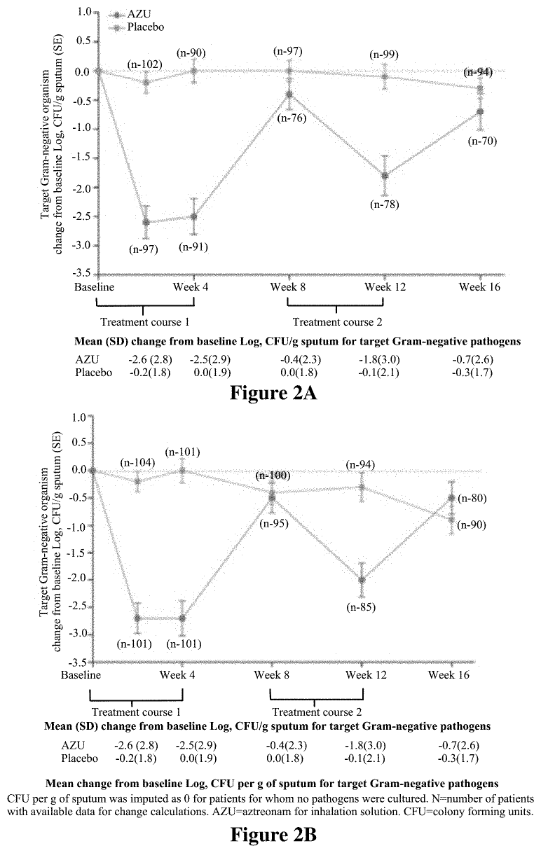

However, the past experience with inhaled antibiotics showed that despite such high concentrations, tolerance to the effect of such inhaled drugs develops. For example, as shown in FIGS. 1-3 for inhaled tobramycin solution (Tobi®), inhaled aztreonam solution Cayston® (AZLI) and inhaled liposomal amikacin aqueous dispersion (Arikayce®, also called Arikace® and Alis®), the antipseudomonal effects of these treatments in patients diminish with time (Tobi: Package insert for inhaled Tobi; Cayston: Barker et al., The Lancet.com/respiratory Vol 2 September 2014, p. 738; Arikace: Clancy et al., Thorax Online First, published on Jun. 8, 2013 as 10.1136/thoraxjnl-2012-202230).

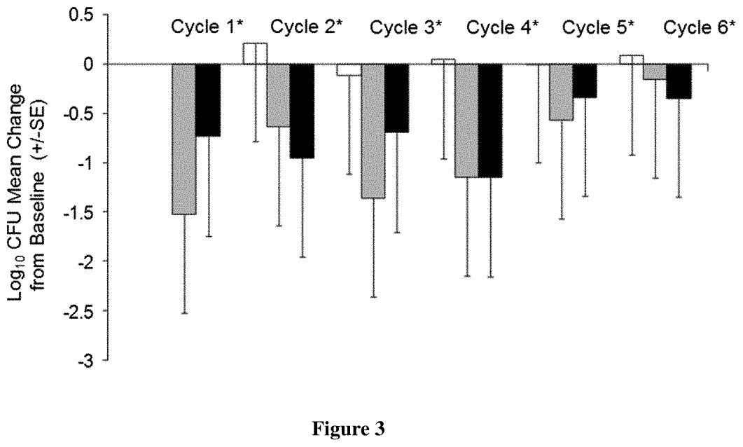

FIG. 3 includes graphs, which show a change in sputum density of Pseudomonas aeruginosa (log10 cfu/g) from baseline through cycle 6 of Arikace. Each set of three bars (white gray and black in FIG. 3) shows the change in sputum P aeruginosa density compared with baseline (day 1 of cycle 1) for days 1 (white), 14 (gray), and 28 (black) of each respective Arikace cycle. *p=0.003 for change in cfu across all of the Arikace treatment cycles relative to baseline (cycle 1, day 1).

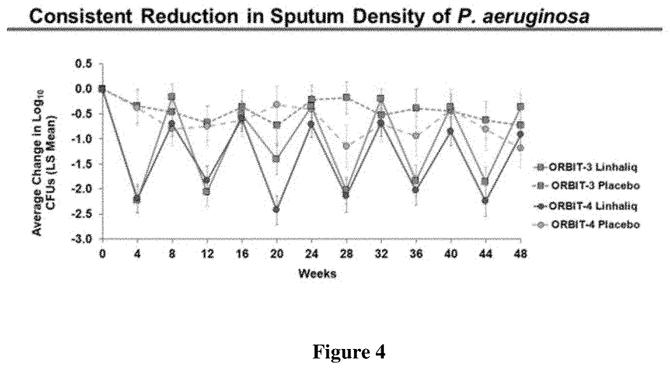

While the exact mechanisms responsible for this behavior are not known, the impact appears to be similar to emergence of resistance: as the treatment continues, the antibiotic effect of these drugs is diminished. Surprisingly and in stark contrast to the above examples, an inhaled formulation contained in this invention comprising of a mixture of liposomally encapsulated ciprofloxacin and unencapsulated ciprofloxacin aqueous solution (Linhaliq®, also called ARD-3150) exhibited undiminished anti-pseudomonal activity during the 48-week double blind period vs. placebo (diluted liposomes in isotonic saline) in the Phase 3 clinical trials in non-cystic fibrosis bronchiectasis patients ORBIT-3 (ARD-3150-1201) and ORBIT-4 (ARD-3150-1202) (FIG. 4) and continued to show the same during the 4 week open label treatment in these trials.

FIG. 4 shows the changes in the average colony forming units (cfus) in the sputum of patients in the ARD-3150 (Linhaliq) vs. Placebo groups

What is notable is that in all of these examples, the inhaled antibiotics were given using the same type of dosage regimens based on repeated cycles of 28 days on treatment followed 28 days off treatment. Although the daily frequency was not the same (Tobi was administered twice daily, Cayston thrice daily, Arikayce once daily—the same as ARD-3150), it is clear that varying the daily frequency was not the differentiating factor but rather the unique properties of ARD-3150 (the proprietary mixture of an aqueous solution of ciprofloxacin and liposomally encapsulated ciprofloxacin).

The ARD-3150 formulation is described below in detail.

An example of a Linhaliq formulation is a mixture of an aqueous solution of ciprofloxacin with ciprofloxacin encapsulated in liposomes.

### Description of Linhaliq

Linhaliq is typically prepared by the patient at time of use by pouring the contents of the two vials, Ciprofloxacin for Inhalation (CFI) and Free Ciprofloxacin for Inhalation (FCI), into the nebulizer.

Linhaliq is a white to off-white translucent dispersion (pH ˜4.5) containing 189 mg ciprofloxacin (as the free base) in 6 ml. The active substance is present as liposomal encapsulated ciprofloxacin (˜70%) and unencapsulated ciprofloxacin solution (˜30%).

Linhaliq is delivered to the respiratory tract using a nebulizer, or another type of delivery system depending on the goal of the therapy, the intended sites of action and the availability of such devices and methods.

### COMPOSITION of Linhaliq

Linhaliq is formed when its two drug product components, CFI and FCI, are added together prior to administration. Typical compositions of Linhaliq are shown below in Tables 1, 2 and 3.

TABLE 1
Composition of Linhaliq

| Drug Product | Concentration |
|---|---|
| Ciprofloxacin | 31.5 |
| Cholesterol | 13.1 |
| Hydrogenated soy | |
| phosphatidylcholine | |
| Ammonium sulphate | 3.5-4.0 (Sulphate); 0.15- |
| Sodium Acetate | 0.65 |
| Histidine | 1.95 |
| Sodium chloride | 4.25 |
| Sulphuric acid | As needed |
| Sodium hydroxide | As needed |
| Glacial acetic acid | As needed |
| Water for | — |

Typical composition of CFI is shown in Table 2:

TABLE 2
Composition of Ciprofloxacin for Inhalation (CFI)

| Component | Quantity |
|---|---|
| Ciprofloxacin | 45.0^{a)} |
| Cholesterol | 26.2 |
| Hydrogenated soy | |
| phosphatidylcholine | |
| Ammonium sulphate | 7-8 Sulfate; 0.3- |
| Histidine | 3.9 |
| Sodium chloride | 8.5 |
| Sulphuric acid | As needed |
| Sodium hydroxide | As needed |
| Water for injection | — |

^{a)}The drug substance added to the formulation is ciprofloxacin hydrochloride, monohydrate at a target 50 mg/mL. The conversion factor from the monohydrate hydrochloride to the free base is 0.90.
^{b}Add sufficient quantity to bring to volume.

Typical composition of FCI is shown in Table 3:

TABLE 3
Composition of Free Ciprofloxacin for Inhalation (FCI)

| Component | Quanity |
|---|---|
| Ciprofloxacin | 18.0^{a} |
| Sodium Acetate | 1. |
| Acetic Acid, Glacial | As needed |
| Sodium | As needed |
| Water for | QS^{b} |

a. ^{a}The drug substance added to the formulation is Ciprofloxacin Hydrochloride, targeting 20 mg/mL. The conversion factor from the hydrochloride to the free base is 0.90.
b. ^{b}Add sufficient quantity to bring to volume.

It is important to appreciate the great clinical importance of reduction of the growth of Pseudomonas aeruginosa (PA) in the lungs of patients with various lung diseases, such as bronchiectasis (BE which is specifically non-cystic fibrosis bronchiectasis, NCFBE). PA is an independent predictor of increased morbidity and mortality, number of pulmonary exacerbations (PEs) and hospitalizations associated with PEs. The course of NCFBE is punctuated by recurrent episodes of PEs, characterized by worsened respiratory symptoms, diminished quality of life, and decreased lung function that result in progressive lung damage, increased rates of hospitalization, and an increased risk of mortality (Chalmers, 2014; Brill, 2015). Chronic infection due to P. aeruginosa is particularly serious, in that it is associated with more extensive disease (as detected by computed tomography), an increased frequency of PEs and hospitalizations, accelerated deterioration of lung function, and a 3-fold increase in mortality (Rayner, 1994; Bilton, 2006; Davies, 2006; Martinez-Garcia, 2007; Wilson, 1997; Scheinberg, 2005; Loebinger,

2009; Noone, 2017; Chalmers, 2014; Martinez-Garcia, 2014; Finch, 2015). The inflammatory load in the lung is related to the PA cfus and its reduction with antibiotics reduces it. A higher bacterial load leads to a higher risk of PEs in patients with bronchiectasis: a 16% increase in the frequency of PEs was found for each 1 log unit increase in cfu/mL in sputum bacterial load, and a larger 25% increase was found for the risk of severe PEs (Chalmers, 2012). The benefit of an inhaled antibiotic that can consistently during chronic therapy keep the cfus of PAs under control, and thus causes reduction of the inflammatory load from the infection, is that it provides the potential for reduction of the frequency of PEs, reduced number of hospitalizations and overall reduced morbidity and mortality.

Treatment with ARD-3150 (Linhaliq) had a greater risk reduction of pulmonary exacerbation (vs placebo) in BE patients who had chronic lung infections with PA. Surprisingly, patients with P. aeruginosa pulmonary infections who developed strains with MICs higher than at baseline during the trial with ARD-3150 (Linhaliq) actually did better than those who did not develop higher MICs. FIG. 5 shows that those who develop MICs above 4 mcg/mL have better response than placebo patients (risk reduction, RR vs. placebo of approximately 0.64) relative to those patients who do not develop MICs above 4 mcg/mL (RR of approximately 0.9). The evidence that this is not a baseline effect is illustrated in the top part of FIG. 5, which shows that the relationship with respect to the baseline MIC is opposite.

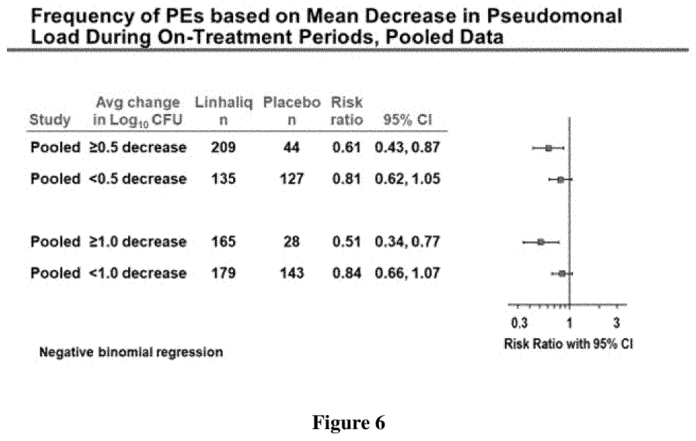

That the beneficial effect is, indeed, due to the reduction of the growth of PA is illustrated in FIG. 6 that shows that those patients who had higher reduction in the colony forming units (cfus) of this microorganism derived a higher benefit in terms of reduction of the frequency of pulmonary exacerbations (PEs).

Negative interactions of inhaled antibiotics with other drugs can be a problem. Inhaled antibiotics are frequently administered to patients who are on other concomitant drugs. Low dose chronic oral macrolide administration has been used to reduce the number of pulmonary exacerbations, for example in diffuse panbronchiolitis, cystic fibrosis, COPD and bronchiectasis. The mechanism of action underlying this use is not well understood; it could be the anti-inflammatory activity of macrolides, or their ability to prevent formation of biofilms or some other mechanism. Macrolides have also antibacterial activity but at the low doses used for prophylaxis of PEs, their concentration is unlikely to have a direct killing or bacteriostatic effect on bacteria such as Pseudomonas aeruginosa that have MICs above the maximum concentrations reached by these macrolide doses.

Interestingly, the most commonly used macrolide azithromycin has been found to be antagonistic against inhaled tobramycin (Nick et al., Ann Am Thorac Soc Vol 11, No 3, pp 342-350, Mar 2014). It is therefore important to develop inhaled antibiotics that do not have antagonistic interaction with concomitant medications. On the contrary, seeing synergy between such treatments would be beneficial.

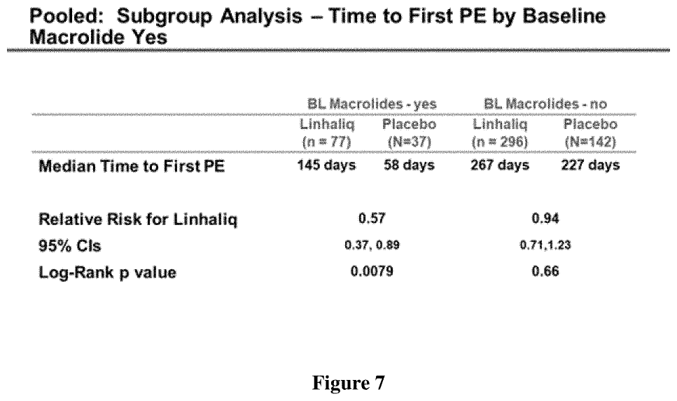

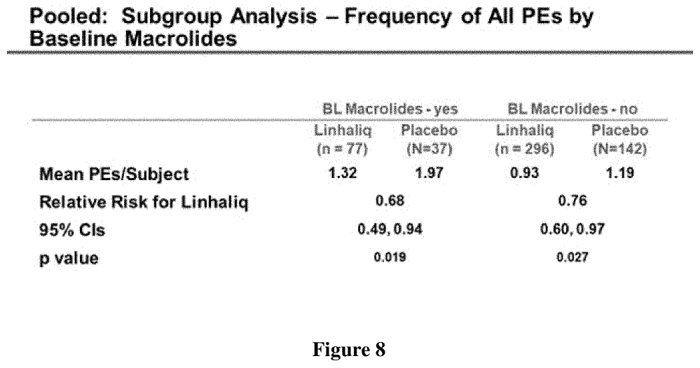

In contrast to the experience with inhaled tobramycin in CF, in the Phase 3 clinical trials with ARD-3150 (Linhaliq) in BE patients with chronic infections with PA who were taking baseline low dose oral macrolides, ARD-3150 compared to placebo had a more profound effect than the therapeutic effect in patients taking ARD-3150 who were not at baseline macrolides, compared to similar patients on placebo. FIGS. 7 and 8 show the pooled data for these phase trials ORBIT-3+ORBIT-4, both showing the time to first pulmonary exacerbation (PE) as well as the frequency of PEs.

It can be observed that there was a more profound impact of ARD-3150 (Linhaliq) in patients on baseline macrolides—relative risk to get the first PE 0.57 (vs placebo) as compared to relative risk of 0.94 for patients not on baseline macrolides (relative risk 0.94). Similarly, the PE frequency reduction relative risk was 0.68 for the population on baseline macrolides taking ARD-3150 vs. 0.76 for those not on macrolides.

ARD-3150 therefore shows benefits irrespective of whether patients were on baseline macrolides, or not. Interestingly, these results also indicate that overall the patients on baseline macrolides were those who had more PEs both in the placebo and ARD-3150 treatment groups compared to patients who were not on baseline macrolides.

This is consistent with another important observation that patients with a severe form of the disease as evidenced by the history of at least 4 PEs in the prior year requiring interventions with antibiotics, benefited from ARD-3150: both the time to first PE in these patients as well as the number of PEs were significantly improved compared to placebo, (these results in favor of ARD-3150 were better than in the subgroup of patients with a lower number of PEs requiring intervention with antibiotics in the prior year—results not shown):

| PE efficacy results by week 48 | ARD-3150 | Placebo (n = 46) |
|---|---|---|
| Number of PEs requiring | 1.07 | 1.78 |
| RR (95% CI); p value | c. 0.56 (0.40, 0.80); p = 0.001 | |
| Median time to 1^{st} PE requiring | 229 | 116 |

| | |
|---|---|
| Treatment effect HR (95% CI); p value | d. 0.60 (0.39, 0.93); p = 0.041 |

It is important to note that this invention also provides a path to treat patients for whom the use of macrolides is a suboptimal option or contra-indicated. High doses of macrolides are a part of the standard treatment of lung infections with non-tuberculous mycobacteria (NTM). The use of low dose macrolides in patients who have concomitant NTM infections or are susceptible to develop such infections is therefore contra-indicated. These concerns exist also for bronchiectasis and CF patients as NTM infections are a relatively common comorbidity in these patients with chronic airway infections with Pseudomonas aeruginosa.

Furthermore, the use of chronic macrolides carries additional risks such as cardiovascular side-effects especially for patients who are at a higher risk from such side-effects; gastrointestinal and hearing loss side-effects are also risks of this medication (Hill, 2016). Therefore, the formulation in this invention can be used as monotherapy when the use of other antibiotics such as macrolides is contra-indicated.

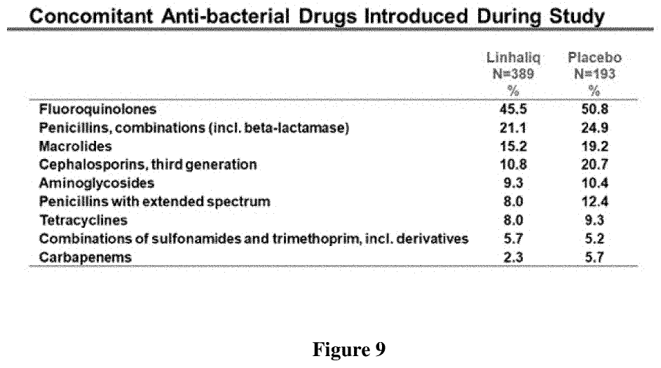

Another surprising observation is that Linhaliq (ARD-3150) use was associated with reduction of emergence of resistance to antibiotics administered systemically (orally or by injection) because the use of ARD-3150 by inhalation generally resulted in lower need for interventions with systemic antibiotics compared to placebo [pooled data from two Phase 3 studies ORBIT-3 (ARD-3150-1201) and ORBIT-4 (ARD-3150-1202)] as shown in FIG. 9.

Another aspect of the invention is to use the emergence of resistance to ciprofloxacin as an indication that the patient may be a good responder to therapy, and the absence of emergence of resistance as a potential non-responder and someone who may need to be coached to inhale properly, adhere to the instructions for storage, use and to the dosage regimen and potentially receive therapy to clear their mucus first with physiotherapy and mucoactive drugs (hypertonic saline, rhDNAse, acetylcysteine, mannitol) and/or bronchodilators to open up the airways to get better penetration of the antibiotic to the lung. In other words, the absence of emergence of bacteria, particularly Pseudomonas aeruginosa, with higher MICs compared to baseline may be an indication of improper use of the formulation and therefore a signal that corrective action should be attempted.

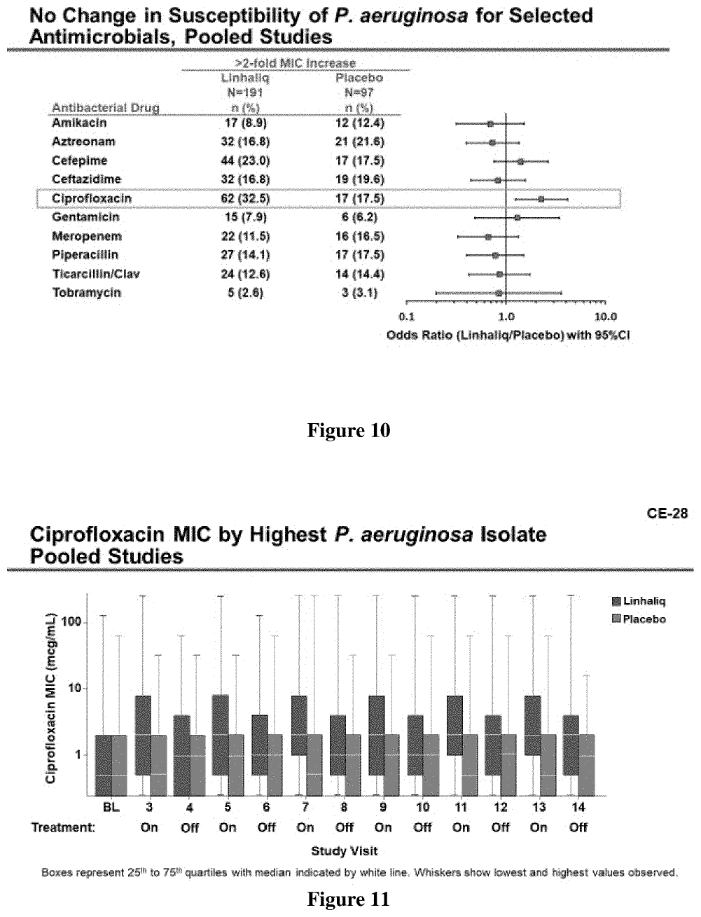

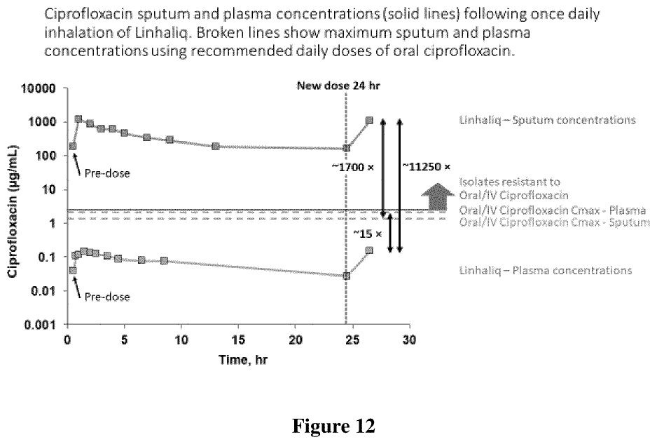

There is a tendency for P aeruginosa in the placebo group compared to the ARD-3150 (Linhaliq) patients to have higher MICs to antibiotics other than ciprofloxacin. i.e., inhalation of ARD-3150 appears to result in the reduction of the use of oral and iv antibiotics and reduced the tendency to increase MICs relative to other antibiotics as shown in FIG. 10. This is another benefit of this invention.

This invention is particularly important for treatment of infections that are resistant to conventional treatments with antibiotics. In particular, this invention is about treating patients who harbor infections, such as respiratory tract infections, that are difficult or impossible to treat with systemic antibiotics, or with other inhaled antibiotics, due to emergence of resistant strains of bacteria such as Pseudomonas aeruginosa, S aureus and mycobacteria including non-TB mycobacteria, or problems with safety and tolerability to other treatments. The methods and the formulations in the invention cause persistent antibacterial effect during treatment periods over a prolonged chronic therapy consisting of periods on treatment and periods off-treatment.

With respect to Pseudomonas aeruginosa, the invention also teaches about the use of absence of reduced susceptibility to the treatment as being a potential sign of non-compliance with the medication.

An aspect of the invention is a formulation which is delivered to the respiratory tract. The formulation contains the first drug which could be present as a mixture of unencapsulated drug (e.g., an anti-infective compound) which may be ciprofloxacin, and the first drug encapsulated in liposomes. The unencapsulated and liposome encapsulated first drug are included within a pharmaceutically acceptable excipient which is formulated for respiratory delivery. The formulation may further include additional therapeutic agents which may be unencapsulated and/or encapsulated and which can be any pharmaceutically active drugs, which are different from the first drug.

Another aspect of this invention is that it can be used as an antibacterial monotherapy against multiple infections. Yet another aspect of this invention is that it can be used initially even if the nature of the infection is not known because of its broad-spectrum antibacterial activity especially against the common serious chronic bacterial infections of the respiratory tract.

The formulation can also be formed in situ during the process of delivery to the respiratory tract, e.g., by formation of an aqueous aerosol. In some situations, the pharmaceutically acceptable carrier can be completely eliminated such as when the unencapsulated drug is in a liquid state. However, the carrier is generally necessary to provide a solvent for the free drug and that solvent may be water, ethanol, a combination of water and ethanol or other useful solvents. The percentage of solvent in the formulation may vary from 0% to 90% but is generally kept at a level, which is sufficiently high to maintain the unencapsulated drug in solution at the pH of the formulation. That level will vary from drug to drug and may vary as the pH varies. The carrier can be present in the formulation in an amount of 10%, 20%, 30%, 40%, 50%, 60% etc. or more or any incremental amounts there in between.

The formulation includes the drug in two different forms. First, the drug is in a free form, which is either liquid or dissolved in a solvent. Second, the drug is encapsulated in liposomes. The ratio of the free drug to the drug encapsulated in liposomes can vary. Generally, the free drug makes up 0%, 5%, 10%, 20%, 30%, etc. up to 80% of the formulation. The drug present within the liposome makes up the remaining percentage of drug present in the formulation. Thus, drug present in the liposomes can be present in the amount of from 20% up to 99% of the total drug present in the formulation.

The formulation may have a pH of 7.4±20%. In some aspects of the invention the formulation is prepared at a relatively low pH such as 5.4 and allowed to adjust to a pH of about 7.4 after it is delivered. Alternatively, the formulation can be formulated at a high pH of about 9.4 and allowed to adjust downward to 7.4 after administration.

The formulation includes liposomes which have the encapsulated pharmaceutically active drug therein which liposomes are designed to provide for controlled release of the drug. Controlled release of this aspect in the invention indicates that the drug may be released at a designed rate to provide a convenient dosage frequency, for example once daily, while still exerting adequate therapeutic effect and not causing undesirable side effects. Ideally therapeutic levels of the antibiotic above the MIC concentration of the most resistant strains of the pathogenic organisms are retained throughout each dosage interval. For example, with once daily dosing, about 50% of the antibiotic or less is released in about 8-12 hours, with most of the antibiotic being released by 24 hours. The rate of release is thus controlled such that an amount of about 0.1% to 100% per hour over a period of time of 1-24 hours or 0.5% to 20% per hour over a period of time of 1-12 hours, or alternatively, releases about 2% to 10% per hour over a period of time of about 1 to 6 hours. Incremental amounts in terms of the percentage of the drug and the number of hours, which are between the ranges provided above in half percentage amounts and half hour amounts and other incremental amounts are intended to be encompassed by the present invention.

One aspect of the invention is a formulation comprising liposomes which are delivered via an aerosol to the lungs of a human patient, the liposomes comprising free and encapsulated ciprofloxacin or another anti-infective agent. The liposomes may be unilamellar or multilamellar, and may be bioadhesive, containing a molecule such as hyaluronic acid. At least one therapeutic agent in addition to the free and liposome-encapsulated anti-infective may also be included in the composition. That therapeutic agent may be free drug or encapsulated drug present with a pharmaceutically acceptable carrier useful for direct inhalation into human lungs. The other drugs may include enzymes to reduce the viscoelasticity of the mucus such as DNase or other mucolytic agents, chemicals to upregulate the chloride ion channel or increase flow of ions across the cells, including lantibiotics such as duramycin, agents to promote hydration or mucociliary clearance including epithelial sodium channel (ENaC) inhibitors or P2Y2 agonists such as denufosol, elastase inhibitors including Alpha-1 antitrypsin (AAT), bronchodilators, steroids, N-acetylcysteine, interferon gamma, interferon alpha, agents that enhance the activity of the antibiotic against biofilm bacteria such as sodium salicylate (Polonio R E et al., 2001), or antibiotics known to those skilled in the art. Inflammation and constriction of the airways are also associated with cystic fibrosis, bronchiectasis, COPD and their treatments. Accordingly, bronchodilators, such as β2-adrenergic receptor agonists and antimuscarinics, and anti-inflammatory agents, including inhaled corticosteroids, non-steroidal anti-inflammatories, leukotriene receptor antagonists or synthesis inhibitors, and others, may also be combined with an anti-infective.

In more recent times, several therapeutic interventions have emerged that correct some of the fundamental defects underlying the various forms of cystic fibrosis related to the genetic mutations causing partial or entire loss of function of the cystic fibrosis transmembrane conductance regulator (CFTR). Such treatments modulate the CFTR function either as monotherapies, or in combination (F. Holguin, NEJM, Oct. 18, 2018; DOI: 10.1056/NEJMe1811996). These drugs are divided into suppressors, correctors and potentiators (Schneider E K et al. Clin Pharmacol Ther. 2017 January; 101(1): 130-141). These, too, can be used in combination with inhaled antibiotics formulated according to the current invention.

The need for better inhaled antibiotics for cystic fibrosis patients is that despite all the existing therapies already available to them, they still carry various respiratory infections that require treatment.

Changes of CFTR—the protein that is defective in CF due to mutations—are also possible as a result of exogenous factors such as tobacco smoking and may therefore lead to disease symptoms similar to cystic fibrosis (Raju et al., Clin Chest Med. 2016 March; 37(1): 147-158). Whether the underlying mechanism in smoking or air-pollution related chronic obstructive disease (COPD) is malfunction of CFTR or some other cause, COPD patients are also susceptible to acquire chronic severe infections with microorganisms such as Pseudomonas aeruginosa and non-tuberculous mycobacteria.

Treatment with antibiotics alone, or in combination with the variety of other drugs mentioned for the treatment of cystic fibrosis may therefore be therapeutic options for these patients, too.

Unfortunately, the same patient may harbor multiple infections and therefore it may be advantageous to use a single treatment that is effective against such co-existing infections. Liposomal ciprofloxacin delivered to the respiratory tract was shown to be effective in animals or humans carrying, for example, lung infections with Pseudomonas aeruginosa, or lung infections with non-tuberculous mycobacteria (Blanchard et al. 2018; Bruinenberg et al., 2010, Cipolla et al. 2016; Serisier et al. 2013). For patients who harbor simultaneously two or more clinically important lung infections, or who are at risk of getting multiple serious lung infections, prophylaxis and treatment with a single liposomal formulation described here may therefore simplify the therapy, reduce the therapeutic burden and the economic costs. For example, the only inhaled antibacterial product approved at present for treatment of pulmonary infections with non-TB mycobacteria (NTM) is the liposomal amikacin for the treatment of refractory lung infections with Mycobacterium avium (Alis, Insmed). About 22% of the patients with pulmonary NTM in US are also co-infected with PA according to a US Registry research (Henkle E. et al., Chest 2017; 152(6):1120-1127). Therefore, it would be advantageous to have a treatment that could be used effectively against this combination of NTM+PA. Staphylococcus aureus (SA) infection is another co-morbidity in patients with chronic PA. For example, in Aradigm's Phase 3 clinical trials ORBIT-3 (ARD-3150-1201) and ARD-3150-1202, this was the most common co-infection occurring in the patients; the treatment with Linhaliq (ARD-3150) tended to reduce the number of these organisms.

A further aspect of the invention is a method for treating patients with one or more respiratory infections, the method comprising administering a formulation comprising the anti-infective; e.g., ciprofloxacin, encapsulated in liposomes to the patient. The formulation is preferably administered by inhalation to the patient for lung infections.

According to another aspect of the present invention, a formulation comprising both a free and encapsulated anti-infective provides an initially high therapeutic level of the anti-infective in the lungs to overcome the bacteria which require high minimum inhibitory concentrations, while maintaining a sustained release of anti-infective over time to allow eradication of organisms that may require either longer exposure for the anti-effective treatment to be effective, or intracellular uptake and/or penetration into biofilms or combination of some or all of these factors. While some aspects of biofilm resistance are poorly understood, the dominant mechanisms are thought to be related to: (i) modified nutrient environments and suppression of growth rate within the biofilm; (ii) direct interactions between the exopolymer matrices, and their constituents, and antimicrobials, affecting diffusion and availability; and (iii) the development of biofilm/attachment-specific phenotypes (Gilbert P et al., 1997). There may be one or more pathogenic infections in a single patient, such as PA and NTM and the formulation in this invention can treat effectively both. Similarly, one or more of the infecting bacteria may be causing intracellular infections, and these, too, can be treated effectively with the formulations in this invention, without the need for the patient to take other drugs to treat the infections. However, sometime, the therapy for the patient may be improved by addition of other drugs, physiotherapy and so on.

The sustained-release anti-infective, e.g., ciprofloxacin, serves to maintain a therapeutic level of antibiotic in the lung thereby providing continued therapy over a longer time frame, increasing efficacy, reducing the frequency of administration, and reducing the potential for resistant colonies to form. Free unencapsulated antibiotic may also serve to attenuate the inflammatory response to the presence of liposomes (i.e., particles) in the lung such as reduction of the influx of macrophages.

Although ciprofloxacin is a particularly useful anti-infective in this invention, there is no desire to limit this invention to ciprofloxacin. Other antibiotics or anti-infectives can be used such as those selected from the group consisting of: an aminoglycoside, a tetracycline, a sulfonamide, p-aminobenzoic acid, a diaminopyrimidine, a quinolone, a beta.-lactam, a beta.-lactam and a .beta.-lactamase inhibitor, chloramphenicol, macrolides, penicillins, cephalosporins, corticosteroid, prostaglandin, linomycin, clindamycin, spectinomycin, polymyxin B, colistin, vancomycin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, terbinafine, or any combination thereof.

These and other objects, advantages, and features of the invention will become apparent to those persons skilled in the art upon reading the details of the formulations and methodology as more fully described below.

### DETAILED DESCRIPTION OF THE INVENTION

Before the present method of formulating ciprofloxacin-encapsulated liposomes and delivery of such for prevention and/or treatment of infections, and devices and formulations used in connection with such are described, it is to be understood that this invention is not limited to the particular methodology, devices and formulations described, as such methods, devices and formulations may, of course, vary. It is also to be understood that the terminology used herein is for the purpose of describing particular embodiments only, and is not intended to limit the scope of the present invention, which will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

As used herein, antibiotic refers to agents that act against infections, or anti-infectives, such as bacterial, viral, fungal, mycobacterial, or protozoal infections. They may belong to specific subclasses, such as anti-bacterials, anti-virals, anti-fungal and so on.

Anti-infectives covered by the invention include but are not limited to quinolones (such as nalidixic acid, cinoxacin, ciprofloxacin and norfloxacin and the like), sulfonamides (e.g., sulfanilamide, sulfadiazine, sulfamethaoxazole, sulfisoxazole, sulfacetamide, and the like), aminoglycosides (e.g., streptomycin, gentamicin, tobramycin, amikacin, netilmicin, kanamycin, and the like), tetracyclines (such as chlortetracycline, oxytetracycline, methacycline, doxycycline, minocycline and the like), para-aminobenzoic acid, diaminopyrimidines (such as trimethoprim, often used in conjunction with sulfamethoxazole, pyrazinamide, and the like), penicillins (such as penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (such as methicillin, oxacillin, cloxacillin, dicloxacillin, nafcillin and the like), first generation cephalosporins (such as cefadroxil, cephalexin, cephradine, cephalothin, cephapirin, cefazolin, and the like), second generation cephalosporins (such as cefaclor, cefamandole, cefonicid, cefoxitin, cefotetan, cefuroxime, cefuroxime axetil, cefinetazole, cefprozil, loracarbef, ceforanide, and the like), third generation cephalosporins (such as cefepime, cefoperazone, cefotaxime, ceftizoxime, ceftriaxone, ceftazidime, cefixime, cefpodoxime, ceftibuten, and the like), other beta-lactams (such as imipenem, meropenem, aztreonam, clavulanic acid, sulbactam, tazobactam, and the like), beta-lactamase inhibitors (such as clavulanic acid), chloramphenicol, macrolides (such as erythromycin, azithromycin, clarithromycin, and the like), lincomycin, clindamycin, spectinomycin, polymyxin B, polymixins (such as polymyxin A, B, C, D, E.sub.1(colistin A), or E.sub.2, colistin B or C, and the like) colistin, vancomycin, bacitracin, isoniazid, rifampin, ethambutol, ethionamide, aminosalicylic acid, cycloserine, capreomycin, sulfones (such as dapsone, sulfoxone sodium, and the like), clofazimine, thalidomide, or any other antibiotic agent that can be lipid encapsulated. Anti-infectives can include antifungal agents, including polyene antifungals (such as amphotericin B, nystatin, natamycin, and the like), flucytosine, imidazoles (such as miconazole, clotrimazole, econazole, ketoconazole, and the like), triazoles (such as itraconazole, fluconazole, and the like), griseofulvin, terconazole, butoconazole ciclopirax, ciclopirox olamine, haloprogin, tolnaftate, naftifine, terbinafine, or any other antifungal that can be lipid encapsulated or complexed and pharmaceutically acceptable salts thereof and combinations thereof. Discussion and the examples are directed primarily toward ciprofloxacin, but the scope of the application is not intended to be limited to this anti-infective. Combinations of drugs can be used.

Bronchodilators covered by the invention include but are not limited to J32-adrenergic receptor agonists (such as albuterol, bambuterol, salbutamol, salmeterol, formoterol, arformoterol, levosalbutamol, procaterol, indacaterol, carmoterol, milveterol, procaterol, terbutaline, and the like), and antimuscarinics (such as trospium, ipratropium, glycopyrronium, aclidinium, and the like). Combinations of drugs may be used.

Anti-inflammatories covered by the invention include but are not limited to inhaled corticosteroids (such as beclometasone, budesonide, ciclesonide, fluticasone, etiprednol, mometasone, and the like), leukotriene receptor antagonists and leukotriene synthesis inhibitors (such as montelukast, zileuton, ibudilast, zafirlukast, pranlukast, amelubant, tipelukast, and the like), cyclooxygenase inhibitors (such as ibuprofen, ketoprofen, ketorolac, indometacin, naproxen, zaltoprofen, lornoxicam, meloxicam, celecoxib, lumiracoxib, etoricoxib, piroxicam, ampiroxicam, cinnoxicam, diclofenac, felbinac, lornoxicam, mesalazine, triflusal, tinoridine, iguratimod, pamicogrel, and the like). Combinations of drugs may be used. Macrolides (such as erythromycin, azithromycin, clarithromycin, and the like) are also used as anti-inflammatory agents, often at doses deemed to be too low to be effective as anti-infective.

Disease modifying therapies, such as the modulators of the CFTR protein (e.g., ivacaftor, lumacaftor or tezacaftor, or a combination of these), can be also used in conjunction with the invention presented here.

As used herein, “Formulation” refers to the liposome-encapsulated anti-infective, with any excipients or additional active ingredients, either as a dry powder or suspended or dissolved in a liquid.

The terms “subject,” “individual,” “patient,” and “host” are used interchangeably herein and refer to any vertebrate, particularly any mammal and most particularly including human subjects, farm animals, and mammalian pets. The subject may be, but is not necessarily under the care of a health care professional such as a doctor.

A “stable” formulation is one in which the active ingredient—a low molecular weight drug, antibody, peptide, protein or enzyme therein essentially retains its physical and chemical stability and integrity upon storage and exposure to relatively high temperatures. Various analytical techniques for measuring peptide stability are available in the art and are reviewed in Peptide and Protein Drug Delivery, 247-301, Vincent Lee Ed., Marcel Dekker, Inc., New York, N.Y., Pubs. (1991), and Jones, A. (1993) Adv. Drug Delivery Rev. 10:29-90. Stability can be measured at a selected temperature for a selected time period.

“Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the claimed methods and compositions.

### Invention in General

Ciprofloxacin is a well-established and extensively utilized broad-spectrum fluoroquinolone antibiotic that is indicated for the treatment of acute lower respiratory tract infections due to P. aeruginosa, which is common in patients with cystic fibrosis and can be a serious chronic infection in other severe lung diseases such as bronchiectasis and chronic obstructive pulmonary diseases (COPD). Unencapsulated ciprofloxacin available as tablets or injections is typically not a drug of choice for the most common non-tuberculous mycobacterial infections in US Mycobacterium avium complex (MAC) due to lack of evidence of efficacy (Aksamit et al., 2014) but in its liposomal form described in this invention, it has been found to be effective in vitro and animal models (Blanchard et al., 2018).

The primary advantage of inhaled antibiotics or specifically antimicrobials is that they target antibiotic delivery to the area of primary infection, bypass GI-related side effects compared to oral antibiotics. They also profoundly change the biodistribution of drugs by having very high concentrations in the respiratory tract for maximum efficacy and relatively low concentrations in the rest of the body to minimize the systemic side effects. However, inhaled delivery is not without its challenges: poor solubility of drugs in the respiratory milieu, local tolerability including bitterness of the drug, short residence time in the respiratory tract and potential for respiratory toxicity due to the high concentrations of the drug or its carriers have limited development of safe and efficacious formulations delivered by inhalation. An example of overcoming some of these obstacles specifically applying to ciprofloxacin is a liposome-encapsulated formulation for the inhalation treatment of pulmonary infections through improved biopharmaceutical characteristics and mechanisms such as increased solubility, flavor masking, altered pharmacokinetics, favorable biodistribution, sustained drug release from the carrier leading to prolonged therapeutic levels with less frequent administration improving patients' experience, enhanced delivery to infections in biofilms and facilitating treatment of intracellular infections.

Preclinical studies demonstrate the efficacy of liposomal ciprofloxacin and the mixtures of unencapsulated ciprofloxacin with liposomal ciprofloxacin, e.g., in a biofilm inhibitory concentration assay using clinical isolates of Pseudomonas aeruginosa, a CF mouse model of P. aeruginosa lung infection; in biofilm, macrophage and rodent models of pulmonary infections with non-tuberculous mycobacteria M avium and M abscessus (Blanchard et al., 2018) and in in vitro and animal models of potential bioterrorism infections such as inhalational tularemia infections (Cipolla et al. 2016).

Safety pharmacology studies of respiration in rats and dogs of inhaled liposomal ciprofloxacin found no marked effects upon respiratory parameters. These studies showed that the inhalation of levels of “free” ciprofloxacin together with the liposomal ciprofloxacin formulation allowed the dosing of greater amounts of liposomal ciprofloxacin without observation of focal macrophage accumulation or increases in the proinflammatory cytokine IL-113 in the lung fluid. This finding is more generally applicable to other liposomal formulations delivered to the lung. These results are also applicable to pharmaceutical preparations or formulations which otherwise may result in increases in macrophage accumulation in the lung or the increase in proinflammatory cytokines in the lung in response to the therapy.

The invention includes a formulation that combines unencapsulated ciprofloxacin (or a different immune blunting agent; e.g., azithromycin) with another drug; e.g., liposomal ciprofloxacin, delivered via the inhalation route, combined with oral macrolide. The liposomal encapsulated ciprofloxacin may be substituted with an antibiotic other than ciprofloxacin and may be formulated with, or without using liposomes, or as a combination of the unencapsulated antibiotic and encapsulated antibiotic. The other drug does not have to be an antibiotic and may be any drug that is believed to have some beneficial properties when delivered to the lung.

Other drugs may include a nucleotide sequence, which may be incorporated into a suitable delivery vector such as a plasmid or viral vector. The other drug may be a therapeutic nucleotide sequence (DNA, RNA, siRNA), enzymes to reduce the viscoelasticity of the mucus such as DNase and other mucolytic agents, chemicals to upregulate the chloride ion channel or increase flow of ions across the cells, nicotine, P2Y2 agonists, elastase inhibitors including Alpha-1 antitrypsin (AAT), N-acetylcysteine, antibiotics and cationic peptides, such as lantibiotics, and specifically duramycin, short-acting bronchodilators (e.g., β2-adrenergic receptor agonists like albuterol or indacaterol), M3 muscarinic antagonists (e.g., ipatropium bromide), K+-channel openers, long-acting bronchodilators (e.g., formoterol, salmeterol), steroids (e.g., budesonide, fluticasone, triamcinolone, beclomethasone, ciclesonide, etc.), xanthines, leukotriene antagonists (e.g., montelukast sodium), phosphodiesterase 4 inhibitors, adenosine receptor antagonists, other miscellaneous anti-inflammatories (e.g., Syk kinase inhibitors (AVE-0950), tryptase inhibitors (AVE-8923 & AVE-5638), tachykinin antagonists (AVE-5883), inducible nitric oxide synthase inhibitors (GW-274150) and others), transcription factor decoys, TLR-9 agonists, antisense oligonucleotides, siRNA, DNA, CGRP, lidocaine, inverse β2-agonists, anti-infective oxidative therapies, cytokine modulators (e.g., CCR3 receptor antagonists (GSK-766994, DPC-168, AZD-3778), TNF-α production inhibitors (LMP-160 & YS-TH2), and IL-4 antagonists (AVE-0309)), small molecule inhibitors of IgE, cell adhesion molecule (CAM) inhibitors, small molecules targeting the VLA4 receptor or integrin α4β1 (e.g., R-411, PS-460644, DW-908e, & CDP-323), immunomodulators including those that block T-cell signaling by inhibition of calcineurin (Tacrolimus), heparin neutralizers (Talactoferrin alfa), cytosolic PLA2 inhibitors (Efipladib), or combinations thereof.

Disease modifying therapies, such as the modulators of the CFTR protein (e.g., ivacaftor, lumacaftor or tezacaftor, or a combination of these), can be also used in conjunction with the invention presented here. The delivery of the combination products may be achieved by combining the drugs into one stable formulation, or providing the drugs in separate containers to be combined at the time of administration or alternatively by sequentially delivering the products.

Exemplary liposome compositions and methods of making them are disclosed in U.S. Pat. Nos. 6,890,555; 6,855,296; 6,770,291; 6,759,057; 6,623,671; 6,534,018; 6,355,267; 6,316,024; 6,221,385: 6,197,333, and 9,968,555 all of which are incorporated herein by reference. The liposomes of the invention may be multilamellar, unilamellar, or any configuration known such as described in the above patents. The liposomes of the instant invention are preferably made from biocompatible lipids. In general, the size of the liposomes generated is over a wide range depending on mode of delivery, e.g. 1 nm to 10 microns or 20 nm to 1 micron or about 100 nm in diameter ±20% for pulmonary delivery, or up to ˜1 micrometer size +/−90%.

### Dosing Regimens

Based on the above, it will be understood by those skilled in the art that a plurality of different treatments and means of administration can be used to treat a single patient. Thus, patients already receiving such medications, for example, oral or intravenous ciprofloxacin and/or other antibiotics, etc., may benefit from inhalation of the formulations of the present invention. Some patients may receive only ciprofloxacin-containing liposome formulations by inhalation. Such patients may have symptoms of cystic fibrosis, bronchiectasis or COPD, be diagnosed as having lung infections, or have symptoms of a medical condition, which symptoms may benefit from administration to the patient of an antibiotic such as ciprofloxacin. The formulations of the invention may also be used diagnostically. In an embodiment, for example, a patient may receive a dose of a formulation of the invention as part of a procedure to diagnose lung infections, wherein one of more of the patient's symptoms improves in response to the formulation. As described above, we have found surprisingly that those patients taking one of the formulations of this invention, ARD-3150 (Linhaliq), to treat their chronic lung infections with PA and developed PA strains in their sputum with elevated MIC of ciprofloxacin compared to their pre-ARD-3150 therapy MIC values overall exhibited a greater reduction of pulmonary exacerbations (PEs) than those patients who did not have elevated MICs. Therefore, measurement of MICs may be used as a diagnostic test, with the elevation of the MICs indicating a likely positive impact of the therapy, while no change in MIC or lower MICs compared to pre-therapy values may suggest that an intervention needs to take place, such as checking the compliance with the therapy, inhalation technique and so on.

This is contrary to the conventional wisdom that elevated MICs result in poorer clinical outcomes with the antibacterial treatment with a drug whose MICs are being tested.

A patient will typically receive a dose of about 0.01 to 10 mg/kg/day of ciprofloxacin However, inhaled antibiotic doses are typically not adjusted for weight. The nominal dose put into the inhalation device depends on several factors, including the target population of patients, nature of the infection and the potency of the drug, and the efficiency of delivery of the medicament to the respiratory tract. For example, in the Phase 3 clinical trials with Linhaliq, the daily loaded dose of ciprofloxacin was 189 mg. This dose will typically be administered by at least one but typically several “puffs” from the aerosol device. The total dose per day is preferably administered as infrequently as possible for the patient's convenience, for example once per day, but may be divided into two or more doses per day. Some patients may benefit from a period of “loading” the patient with a higher dose or more frequent administration over a period of days or weeks, followed by a reduced or maintenance dose. As such infections may be chronic, patients are expected to receive such therapy over a prolonged period of time. It is also quite customary that such inhaled antibiotic therapies are taken for a certain period of time, and then the patient stops using them and restarts later. Cycles of 28 days on inhaled antibiotics followed by 28 days off the medication are well established for the treatment of chronic respiratory infections with Pseudomonas aeruginosa cystic fibrosis patients. Other such combinations on/off therapeutic cycles have been also tested, for example with inhaled dry powder formulation of ciprofloxacin for the treatment of chronic lung infections in patients with non-cystic fibrosis bronchiectasis (DeSoyza et al. 2018; Aksamit et al., 2018). Continuous treatment has been also used with inhaled colistin in cystic fibrosis and NCFBE patients with P. aeruginosa lung infections (e.g., Haworth et al., 2014) and it may be a beneficial regimen if the formulation performs as Linhaliq, i.e., shows persistent antimicrobial activity over a prolonged period of treatment.

It has previously been shown that inhalation of liposome-encapsulated fluoroquinolone antibiotics may be effective in treatment of lung infections and were shown to be superior to the free or unencapsulated fluoroquinolone in a mouse model of F. tularensis (CA 2,215,716, CA 2,174,803 and CA 2,101,241). Another application, EP1083881B1, describes liposomes containing a drug-conjugate comprising a quinolone compound covalently attached to an amino acid.

Another application, US 2000142026, also describes the use of liposome-encapsulated antibiotics. That application discusses the potential for administration of a lower dose of a liposome-encapsulated antibiotic, by a factor of 10 or 100, than for the free unencapsulated antibiotic. Other applications describing the use of liposomes containing anti-infectives to treat cystic fibrosis, US 20060073198 and US 20040142026 show efficacy for a lower dose liposomal formulation compared to the use of the free drug alone.

Thus, as discussed above, the formulations according to some aspects of the invention include free or non-encapsulated ciprofloxacin in combination with the liposome-encapsulated ciprofloxacin. Such formulations may provide an immediate benefit with the free ciprofloxacin resulting in a rapid increase in the antibiotic concentration in the lung fluid surrounding the bacterial colonies or biofilm and reducing their viability, followed by a sustained benefit from the encapsulated ciprofloxacin which continues to kill the bacteria or decrease its ability to reproduce, or reducing the possibility of antibiotic resistant colonies arising. The skilled practitioner will understand that the relative advantages of the formulations of the invention in treating medical conditions on a patient-by-patient basis.

### Combination Therapies

Liposome formulations of the invention may be administered concurrently with other drugs as described here. For example, the liposomes of the invention may be used along with drugs such as CFTR modulators, hyaluronic acid, DNase, a mucolytic agent, chemicals that up-regulate the chloride ion channel or increase flow of ions across the epithelial surface of cells, a bronchodilator, a steroid, a P2Y2 agonist, an elastase inhibitor such as Alpha-1 antitrypsin (AAT), N-acetylcysteine, agents that enhance the activity of the antibiotic against biofilm bacteria such as sodium salicylate, interferon gamma, interferon alpha, or a fluoroquinolone selected from the group consisting of amifloxacin, cinoxacin, ciprofloxacin, danofloxacin, difloxacin, enoxacin, enrofloxacin, fleroxacin, irloxacin, lomefloxacin, miloxacin, norfloxacin, ofloxacin, pefloxacin, rosoxacin, rufloxacin, sarafloxacin, sparfloxacin, temafloxacin and tosufloxacin or an antibiotic selected from the group of tobramycin, colistin, azithromycin, amikacin, cefaclor (Ceclor), aztreonam, amoxicillin, ceftazidime, cephalexin (Keflex), gentamicin, vancomycin, imipenem, doripenem, piperacillin, minocycline, or erythromycin. Chronic therapy with macrolides (such as erythromycin, azithromycin, clarithromycin, and the like) is also used as it is thought to be reducing pulmonary exacerbations, possibly through anti-inflammatory mechanisms.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### Method of Treatment

This method of treatment applies to prophylaxis and therapies for disease states which involve infections, not limited to but including those of the nasal passages, airways, ears, or lungs. These infections can be found in a variety of otherwise healthy subjects or patients with sinusitis, cystic fibrosis, bronchiectasis, chronic pulmonary obstructive disease, pulmonary non-tuberculous mycobacteria, tuberculosis and infections that could be potential bioterrorism weapons such as inhalational tularemia, inhalational anthrax, pneumonic plague, Q-fever, melioidosis, glanders; pneumonia including but not limited to ventilator associated pneumonia, community acquired pneumonia, bronchial pneumonia, lobar pneumonia; infections by Streptococcus pneumoniae, Chlamydia, Mycoplasma pneumonia, staphylococci. Prophylactic treatment or prevention for conditions in which infection might arise include, e.g., intubated or ventilated patients, infections in lung transplant patient, bronchitis, pertussis (whooping cough), inner ear infections, or streptococal throat infections. It is a major advantage of the inhaled liposomal formulations of ciprofloxacin and similar broad-spectrum antibiotics that they could be used in an emergency situation before the exact nature of the infection is identified, or even before the first symptoms occur if it is plausible that a person was exposed to air containing dangerous microorganisms.

The response of the patient to a formulation of our invention can be also used to optimize the treatment. For example, emergence of more resistant PA following the treatment with one of our formulations ARD-3150 (Linhaliq) was associated with a higher therapeutic efficacy in terms of reduction of the number of pulmonary exacerbations. As would be appreciated, often the detailed nature of all of the underlying infections with pathogenic organisms is not known; positive response to the therapy may therefore assist in narrowing down the nature of the infections causing the symptoms of the disease. A related aspect of the invention is that the formulations of our invention due to their ability to treat successfully a multitude of infections can be used in situations where the exact nature of the infections is not known, e.g., when pathogenic airborne infections may be used as weapons or are spread inadvertently by air currents.

### REFERENCES

Each of the following is incorporated by reference.
Abbey D E, Hwang B L, Burchette R J, Vancuren T, Milss P K. Estimated Long-Term Ambient Concentrations of PM10 and Development of Respiratory Symptoms in Nonsmoking Population. Arch Environ Health 1995; 50(2): 139-151.
Almeida J A, Runge S, Miilleret R H, Peptide-loaded solid lipid nanoparticles (SLN): Influence of production parameters. Int J Pharm. 149 (2) (1997) 255-265.
Bangham A D, Standish M M, Watkins J C, Diffusion of univalent ions across the lamellae of swollen phospholipids. J Mol Biol. 13 (1) (1965) 238-252.
Barker et al., The Lancet.com/respiratory Vol 2 September 2014, p. 738.
Bilton et al. Addition of Inhaled Tobramycin to Ciprofloxacin for Acute Exacerbations of Pseudomonas aeruginosa Infection in adult Bronchiectasis, CHEST, 2006, Vol. 130 (5), 1503-1510.
Blanchard et al., Effective Treatment of Mycobacterium avium subsp. Hominissuis and Mycobacterium abscessus Species Infections in Macrophages, Biofilm, and Mice by Using Liposomal Ciprofloxacin. Antimicrobial Agents and Chemotherapy Sep. 2018, 62 (10) e00440-18.
Brill et al., Lung Functoin, symptoms and inflammation during exacerbations of non-cystic fibrosis bronchiectasis: a prospective observational cohort study. Resp Res. 2015; 16: 1-9.
Bruinenberg et al., Inhaled liposomal ciproflocacin: Once a day management of respiratory infections. Respiratory Drug Delivery, 2010, 1, 73-82.
Chalmers et al., Short- and long-term antibiotic treatment reduces airway and systemic inflammation in non-cystic Am J Respir Crit Care Med 2012; 186, 657-665.
Chalmers et al., The Bronchiectasis Severity Index. An International Derivation and Validation Study. AJRCCM. 2014; 189: 576-585; 2014
Choudhury A, Gordian M E, Morris S. Associations Between Respiratory Illness and PM10 Air Pollution. Arch Environ health 1997; 54(2): 113-117.
Cipolla et al. Development of Liposomal Ciprofloxacin to Treat Lung Infections, Pharmceutics, Mar. 2016, 8(1): 6.
Clancy et al., Phase II studies of nebulized Arikace in CF patients with Psuedomonas aeruginosa infection. Thorax Online First, published on Jun. 8, 2013 as 10.1136/thoraxjnl-2012-202230.
Conforti A, Franco L, Milanino R, Velo G P, Boccu E, Largajolli E, Schiavon O, Veronese F M. PEG superoxide dismutase derivatives: anti-inflammatory activity in carrageenan pleurisy in rats. Pharm Research Commun. 19, pg. 287, 1987.
Costerton J W, Lewandowski Z, Caldwell D E, Korber D R, Lappin-Scott H M., Microbial biofilms. Annu Rev Microbiol. 1995; 49:711-45.
Davies et al., The effect of Pseudomonas aeruginosa on pulmonary function in patients with bronchiectasis. Eur. Respir. J. Nov. 2006, 28 (5) 974-9.
Eric Wenzler, Dustin R. Fraidenburg, Tonya Scardina, Larry H. Danziger, Clin. Microbiol. Rev. 2016 Volume 29 Number 3 p.581.
Finch et al., A Comprehensive Analysis of the Impact of Pseudomonas aeruginosa Colonization on Prognosis in Adult Bronchiectasis. Ann. Am. Thorac. Soc. Nov. 2015; 12(11): 1602-11.
Fitzsimmons S C. The changing epidemiology of cystic fibrosis. J Pediatr. 1993 January; 122(1):1-9.
Gilbert P, Das J, Foley I., Biofilm susceptibility to antimicrobials. Adv Dent Res. 1997 April; 11(1):160-7.
Haworth C S, Foweraker J E, Wilkinson P, et al. Inhaled colistin in patients with bronchiectasis and chronic Pseudomonas aeruginosa infection. Am J Respir Crit Care Med. 2014 Apr. 15; 189:975-82.
Helle J, Barr J, Ng S Y, Shen H R, Schwach-Abdellaoui K, Gurny R, Vivien-Castioni N, Loup P J, Baehni P, Mombelli A. Development and applications of injectable poly(ortho esters) for pain control and periodontal treatment. Biomaterials, 23, 2002, 4397-4404.
Henkle E. et al., Pharmacotherapy for Non-Cystic Fibrosis Bronchiectasis: Results from an NTM Info & Research Patient Survey and the Bronchiectasis and NTM Research Registry. Chest 2017; 152(6):1120-1127
Hodson M, Shah P L, DNase trials in cystic fibrosis. Respiration 1995; 62, Suppl 1:29-32.
Katre N V, Knauf M J, Laird W J., Chemical modification of recombinant interleukin 2 by polyethylene glycol increases its potency in the murine Meth A sarcoma model. Proc Natl Acad Sci. U.S.A. vol. 84, 1487-91, 1987.
Koren H. Association between Criteria Air Pollutants and Asthma. Environ Health Perpec 1995; 103(6): 235-242.
Lode H, Borner K, Koeppe P, Schaberg T., Azithromycin--review of key chemical, pharmacokinetic and microbiological features. J Antimicrob Chemother. 1996; 37, Suppl C: 1-8
Loebinger, Mortality in bronchiectasis: a long-term study assessing the factors influencing survival. ERJ Express, 2009.
Martinez-Garcia, Factors Associated With Lung Function Decline in Adult Patients with Stable Non-Cystic Fibrosis Bronchiectasis. November 2007, Chest, Vol. 132 (5): 1565-1572.
Martinez-Garcia, Multidimensional approach to non-cystic fibrosis bronchiectasis: the FACED score. 2014, ERJ, 43:1357-1367.
Moss R B., Administration of aerosolized antibiotics in cystic fibrosis patients. Chest. 2001 September; 120(3 Suppl):107S-113S.
Nick et al., Azithromycin May Antagonize Inhaled Tobramycin When Targeting Pseudomonas aeruginosa in Cystic Fibrosis. Ann Am Thorac Soc Vol 11, No 3, pp 342-350, Mar 2014.
Niven R N and Schreier H. Nebulization of Liposomes: I. Effects of Lipid Composition. Pharm Res. 1990; 7:1127-1133.
Noone, Healthcare Cost and Utilization before and after Diagnosis of Psuedomonas aeruginosa among Patients with Non-Cystic Fibrosis Bronchiectasis in the U.S. Med. Sci. (Basel) 2017 Sep. 23; 5(4).
Nurkiewicz T R, Porter D W, Barger M, Castranova V, Boegehold M A: Particulate matter exposure impairs systemic microvascular endothelium-dependent dilation. Environ Health Perspect 2004, 112:1299-1306
Nurkiewicz T R, Porter D W, Hubbs A F, Cumpston J L, Chen B T, Frazer D G, Castranova V., Nanoparticle inhalation augments particle-dependent systemic microvascular dysfunction. Particle and Fibre Tox., 2008. 5:1.
Passirani C, Barratt G, Devissaguet J P, Labarre D., Long-circulating nanoparticles bearing heparin or dextran covalently bound to poly (methyl methacrylate). Pharm Res. 1998 July; 15(7):1046-50.
Peppas Bures P, Leobandung W, Ichikawa H., Hydrogels in pharmaceutical formulations. Eur J of Pharm and Biopharm. 2000 July; 50(1):27-46. Review.
Polonio R E Mermel L A, Paquette G E, Sperry J F., Eradication of biofilm-forming Staphylococcus epidermidis (RP62A) by a combination of sodium salicylate and vancomycin. Antimicrob Agents Chemother. 2001 November; 45(11):3262-6.
Pope C A, Bates D, Raizenne M. Health Effects of Particulate Air Pollution: Time for Reassessment? Environ Health Perspec 1995; 103: 472-480.
Raju et al., Acquired CFTR Dysfunction in Chronic Bronchitis and Other Diseases of Mucus Clearance. Clin Chest Med. 2016 March; 37(1): 147-158.
Rayner, Efficacy and Safety of long-term ciprofloxacin in the management of severe bronchiectasis. J. Antimicrob. Chemomother. 1994 Jul, 34(1): 149-156.
Scheinberg, A pilot study of the safety and efficacy of tobramycin solution for inhalation in patients with sever bronchiectasis. 2005
Schneider E K et al. Can Cystic Fibrosis Patients Finally Catch a Breath With Lumacaftor.Ivacaftor? Clin Pharmacol Ther. 2017 January; 101(1): 130-141
Schroeder S A Gaughan D M, Swift M., Protection against bronchial asthma by CFTR delta F508 mutation: a heterozygote advantage in cystic fibrosis. Nat Med.1995 July; 1(7):703-5.
Sciarra J J, Patel S P., In vitro release of therapeutically active ingredients from polymer matrixes. J of Pharm Sci. 1976 October; 65(10):1519-22.
Serisier et al. Inhaled, dual release liposomal ciprofloxacin in non-cystic fibrosis bronchiectasis (ORBIT-2): a randomized, double-blind, placebo-controlled trial. Thorax. 2013 September; 68 (9): 812-7.
Smith A L, Ramsey B W, Hedges D L, Hack B, Williams-Warren J, Weber A, Gore E J, Redding G J. Safety of aerosol tobramycin administration for 3 months to patients with cystic fibrosis. Ped Pulmonol. 1989; 7(4):265-271.
Thorgeirsdottir T O, Kjoniksen A L, Knudsen K D, Kristmundsdottir T, Nystrom Viscoelastic and Structural Properties of Pharmaceutical Hydrogels Containing Monocaprin. Eur. J. of Pharm. and Biopharm. 2005 February; 59(2):333-42.

Van Heeckeren A M, Schluchter M D, Drumm M L, Davis P B. Role of Cftr genotype in the response to chronic Pseudomonas aeruginosa lung infection in mice. Am J Physiol Lung Cell Mol Physiol. 2004 November; 287(5):L944-52. Epub 2004 July 9.
Van Heeckeren A M, Scaria A, Schluchter M D, Ferkol T W, Wadsworth S, Davis P B. Delivery of CFTR by adenoviral vector to cystic fibrosis mouse lung in a model of chronic Pseudomonas aeruginosa lung infection. Am J Physiol Lung Cell Mol Physiol. 2004 April; 286(4):L717-26. Epub 2003 Sep. 26.
Van Heeckeren A, Ferkol T, Tosi M., Effects of bronchopulmonary inflammation induced by Pseudomonas aeruginosa on adenovirus-mediated gene transfer to airway epithelial cells in mice. Gene Ther. 1998 March; 5(3):345-51.
Van Heeckeren A M, Schluchter M D., Murine models of chronic Pseudomonas aeruginosa lung infection. Lab Anim. 2002 July; 36(3):291-312.
Van Heeckeren A M, Tscheikuna J, Walenga R W, Konstan M W, Davis P B, Erokwu B, Haxhiu M A, Ferkol T W. Effect of Pseudomonas infection on weight loss, lung mechanics, and cytokines in mice. Am J Respir Crit Care Med. 2000 January; 161(1):271-9. Weber A, Smith A, Williams-Warren J, Ramsey B, Covert D S., Nebulizer delivery of tobramycin to the lower respiratory tract. Pediatr Pulmonol. 1994 May; 17(5):331-9.
Wilson, Incidence of Pseudomonas aeruginosa bacteremia: a population-based study. Am J Med. 1997 August; 121(8): 702-8.
Wolter J, Seeney S, Bell S, Bowler S, Masel P, McCormack J., Effect of long term treatment with azithromycin on disease parameters in cystic fibrosis: a randomised trial. Thorax 2002; 57: 212-216.
Woodle M C, Storm G, Newman M S, Jekot J J, Collins L R, Martin F J, Szoka F C Jr., Prolonged systemic delivery of peptide drugs by long-circulating liposomes: illustration with vasopressin in the Brattleboro rat. Pharm Res.1992 February; 9(2):260-5.
Zielenski J, Tsui L C., Cystic fibrosis: genotypic and phenotypic variations. Annual Rev Genet. 1995; 29:777-807.
Ye, Q, Asherman J, Stevenson M, Brownson E, Katre N V., DepoFoam technology: a vehicle for controlled delivery of protein and peptide drugs. J Control Rel. February 14; 64(1-3):155-66.
Yevich P P, Lung structure—relation to response to particulate. Arch. Environ. Health 10: 37-43 (1965).

What is claimed is:

1. A method of treatment, comprising:
a) diagnosing a subject with bronchiectasis and chronic respiratory bacterial infections, with at least one strain of Pseudomonas aeruginosa bacteria in an airway sample taken from the subject;
b) administering to the subject over a plurality of days by inhalation a formulation comprised of the un-encapsulated and encapsulated antibiotic;
c) discontinuing the administering over a plurality of days;
d) repeating the administering over a plurality of days;
e) whereby an antibacterial impact of the formulation on the Pseudomonas aeruginosa is decreased by 10% or less with each repeating step (d).

2. The method of claim 1, wherein the antibiotic is ciprofloxacin and the encapsulation is in liposomes, and in view of (e) dosing is not increased.

3. The method of claim 2, wherein the ciprofloxacin is present in solution in an unencapsulated form and in solution in an encapsulated form at concentration in a range of from about 10 mg per milliliter to 40 mg per milliliter.

4. The method of claim 3 wherein the plurality of days is selected from the group consisting of 5 days, 10 days, 14 days, 21 days, and 28 days wherein the plurality of days is days for administering in step (b), discontinuing the administering in step (c) and repeating the administering in step (d).

5. The method as claimed in claim 4, wherein the subject has an immediate history of treatment with chronic macrolide therapy and the macrolide is selected from the group consisting of:
Azithromycin, Clarithromycin, Erythromycin, and Roxithromycin,
wherein the administering is carried out by aerosolizing the formulation and inhaling the aerosol in the patient's lungs, whereby 90% or more of the liposome maintain their structural integrity when aerosolized;
wherein the liposomes are comprised of cholesterol and hydrogenated soy phosphatidyl choline (HSPC); and
wherein the repeating step (d) is carried out until the respiratory infection with Pseudomonas aeruginosa is no longer detected in the patient for at least 2 months.

6. The method of claim 5, wherein the ciprofloxacin inside the liposomes at a concentration of 50% or more higher than a concentration of the unencapsulated ciprofloxacin solution;
wherein the formulation further comprises a buffer selected from the group consisting of sodium acetate and histidine;
wherein the highest minimum inhibitory concentration (MIC) of ciprofloxacin to Pseudomonas aeruginosa remains unchanged with each repeating step (d).

7. The method of claim 6, wherein the ciprofloxacin is present in the liposomes at a concentration sufficiently high that ciprofloxacin precipitates out of solution, and the precipitate dissolves when delivered to a patient after the liposomes open.

8. A method of treatment, comprising:
a) diagnosing a subject with bronchiectasis and chronic respiratory infections with Pseudomonas aeruginosa;
b) taking an airway sample such as sputum, throat swab or bronchoalveolar lavage no more than 1 month prior to the first dose of the treatment and testing the minimum inhibitory concentration (MIC) of Pseudomonas aeruginosa in these samples against ciprofloxacin;
c) administering to the subject by inhalation a formulation comprised of un-encapsulated and encapsulated ciprofloxacin;
d) repeating the administering daily over a period of weeks;
e) taking a second airway sample or multiple samples such as sputum, throat swab or bronchoalveolar lavage no less than 1 week after commencement of the first dose of the treatment but not after the treatment has been discontinued for more than 3 days, and measuring the MICs of Pseudomonas aeruginosa in these samples against ciprofloxacin;
f) comparing the MICs in the samples from (b) and (e);
g) checking patient for compliance with the instructions for the storage and use of the treatment if at least one of the MICs in (e) is not higher than the highest MIC in (b) and ascertaining correct storage and use by the subject.

9. The method of claim 8, wherein the administering is carried out by aerosolizing the formulation and inhaling the aerosol in the patient's lungs, whereby 90% or more of the liposome maintain their structural integrity when aerosolized;
wherein the liposomes are comprised of cholesterol and hydrogenated soy phosphatidyl choline (HSPC);
wherein the repeating step (d) is carried out until the respiratory infection with Pseudomonas aeruginosa is no longer detected in the patient for at least 2 months;
wherein the ciprofloxacin inside the liposomes at a concentration of 50% or more higher than a concentration of the unencapsulated ciprofloxacin solution; and
wherein the formulation further comprises a buffer selected from the group consisting of sodium acetate and histidine.

10. The method of claim 8, wherein the highest minimum inhibitory concentration (MIC) of ciprofloxacin to Pseudomonas aeruginosa remains unchanged with each repeating step (d); and
wherein the ciprofloxacin is present in the liposomes at a concentration sufficiently high that ciprofloxacin precipitates out of solution, and the precipitate dissolves when delivered to a patient after the liposomes open.

11. A method of treatment, comprising:
a) diagnosing a subject with a refractory bacterial infection in a body cavity previously treated unsuccessfully, wherein the patient is further determined to have a characteristic selected from the group consisting of: (1) lack of compliance with prescribed dosing regimen; (2) improper delivery technique; (3) problems with the delivery device used in treatment; (4) lack of drug stability and/or formulation stability; (5) obstruction causing failure of formulation to enter the site of infection;
b) rectifying any of the characteristics 1-5 pertinent to the patient
c) administering directly to the infection in the body cavity a formulation comprised of an un-encapsulated and encapsulated antibiotic.

12. The method of claim 11, wherein the antibiotic is ciprofloxacin, and wherein the bacteria is Pseudomonas aeruginosa, in the encapsulation is in liposomes; and
wherein the body cavity is the respiratory tract.

13. The method of claim 11, further comprising:
repeating the administering each day for 28 days;
discontinuing the administering for 28 days; and
repeating the administering each day for an additional 28 days.

14. The method of claim 11, wherein the administering is carried out by aerosolizing the formulation and inhaling the aerosol in the patient's lungs, whereby 90% or more of the liposome maintain their structural integrity when aerosolized.

15. A method of treatment, comprising
a) diagnosing a subject with an infection in a body cavity;
b) testing a sample from an area of the infection and determining a minimum inhibitory concentration (MIC) of antibiotic to inhibit growth of bacteria in the infection;
c) administering directly to the infection in the body cavity a formulation comprised of un-encapsulated and encapsulated antibiotic, wherein the concentration of the antibiotic is from two times to 1000 times the MIC determined in the testing in b).

16. The method of claim 15, wherein the antibiotic is ciprofloxacin, the encapsulation is in liposomes, the body cavity is the respiratory tract;
the method further comprising:
repeating the administering each day for 28 days;
discontinuing the administering for 28 days; and
repeating the administering each day for an additional 28 days.

17. A method of treatment, comprising:
administering directly to the bacterial infection in the body cavity a formulation comprised of a solution of antibiotic having therein an encapsulated solution of antibiotic;
repeating the administering each day for 28 days; discontinuing the administering for 28 days; and
repeating the administering each day for an additional 28 days.

18. The method of claim 17, wherein the antibiotic is ciprofloxacin, and wherein the bacterial infection is caused by Pseudomonas aeruginosa;
the encapsulation is in liposomes.
the body cavity is the respiratory tract.

19. A method of treatment and diagnosis of respiratory infections comprising of:
a) measuring the susceptibility of microorganisms in samples from the respiratory tract to the antibiotics used in the formulation before the treatment in claim 17 commences;
b) measuring the susceptibility of microorganisms in samples from the respiratory tract to the antibiotics used in the formulation, after the formulation in claim 17 has been administered at least for 3 days;
c) comparing the susceptibilities measured in a) and b);
d) check the patient's compliance with the instructions for the use of the medication if the minimum inhibitory concentrations in b) is not higher than in a).

20. The method of treatment in claim 19 whereby the patient's compliance with the instructions for use is determined by analyzing in the samples from respiratory tract, and the patients selected for treatment have experienced multiple pulmonary exacerbation in the prior year while receiving treatment and/or prophylaxis.
